(19) United States
(12) Patent Application Publication
(10) Pub. No.: US 2008/0280318 A1
(43) Pub. Date: Nov. 13, 2008

(54) Isolation and Culture of a High Purity Population of Cone Photoreceptor Cells by Tissue Dissociation and Pna-Panning, and Biological Applications Thereof

(75) Inventors: Elise Balse, Paris (FR); Luc-Henri Te ssier, Strasbourg (FR); Celine Fuchs, Erstein (FR); Valerie Forster, Paris (FR); Jose-Alain Sahel, Paris (FR); Serge Picaud, Avon (FR)

Correspondence Address: MARSHALL, GERSTEIN & BORUN LLP, 233 S. WACKER DRIVE, SUITE 6300, SEARS TOWER, CHICAGO, IL, 60606, US

(73) Assignee: INSERM (Institut National de la Santé et de la Recherche Médicale), Paris (FR)

(21) Appl. No.: 11/587,123

(22) PCT Filed: Apr. 22, 2005
(86) PCT No.: PCT/EP2005/005242

§ 371 Date: Oct. 20, 2006

(30) Foreign Application Priority Data
Apr. 23, 2004 (EP) 04291067.9

(51) Int. Cl. C12Q 1/02; C12N 5/02; C12N 5/06; C12N 5/08

(52) U.S. Cl. 435/29; 435/395; 435/378; 435/379; 435/404; 435/353; 435/354; 435/366; 435/325; 435/308.1

(57) ABSTRACT

The present invention provides a process and kit for isolating cone photoreceptor cells from retinal tissue with a purity level of at least 80%, typically of about 90%. The isolation process uses a PNA-panning procedure conducted on dissociated retinal tissue. The present invention also provides a culture medium enabling the in vitro survival and development of such isolated cone cells. The means of the invention are applicable to adult mammalian cone cells, and more particularly to adult human cone cells. They have the advantage of being applicable to pathologic or otherwise altered cone cells, and thus give access to the screening of compounds capable of showing neuroprotective activity on adult cone cells.

### FIELD OF THE INVENTION

The present invention generally relates to the field of cone photoreceptor cell production by isolation from a retinal tissue, and to the biological applications thereof, notably as screening tools to identify compounds capable of showing anti-degenerative properties on cone photoreceptors.

### BACKGROUND OF THE INVENTION

Photoreceptor degeneration is the cause of blindness in diseases such as retinitis pigmentosa and age macular degeneration. In these diseases, the cause of blindness or low vision is due to cone photoreceptor degeneration. Night blind people can lead a normal life especially in industrial countries and useful vision can still prevail despite a 95% loss of cones (Geller and Sieving, 1993). Preventing cone cell loss thus appears as a main target in therapeutic strategies. In retinitis pigmentosa, although most causative mutations affect rod photoreceptor specific genes, rod apoptosis is followed by a secondary cone degeneration. This secondary cone photoreceptor degeneration was attributed to a rod-dependent trophic factor required for cone survival (Mohand-said et al., 1998; Fintz et al., 2003).

All these results and considerations urged the need to screen cone photoreceptor survival factors, however this screening was limited by the lack of a pure photoreceptor cell culture.

There have already been some attempts in this respect, but they were either limited to non-adult cells (e.g. embryo retinal cells), and/or non applicable to mammalian cells, and/or did not achieve a sufficiently pure degree of cone isolation.

For example, cell suspensions from 8-d chick embryo retina have been cultured in vitro for up to 7 days in low-density glial-free monolayers, and thereby led to a partial differentiation into developing chick cones (Adler et al., 1984; Adler, 1990; Fintz et al., 2003). The chick embryo retinal cell cultures obtained therefrom could reach a purity of up to 70%. However such cultures may not be representative of the physiology and biochemistry of an adult cone cell. They also cannot provide information about diseased adult cone cells.

Cell cultures containing adult mammalian retinal cells have also been produced, but they included both cone and rod photoreceptors (Hicks et al., 1994; Gaudin et al., 1997; Picaud et al, 1998a). When the retina was sectioned in its thickness to isolate the photoreceptor layer, rods and cones survived only on a glial feeder cell layer (Picaud et al., 1998b). This approach therefore did not allow to separate rods from cones. It however provided evidence for a strict photoreceptor dependence on glial cells. Nevertheless the rod dependence for cone survival could not be addressed on this model.

Another method of progressive dissociation has been described that provided relatively pure photoreceptor cell culture, but still in a rod-cone ratio of 2:3.1 (Traverso et al., 2003). This preparation was used to confirm that trophic factors like EGF and FGF2 can postpone photoreceptor degeneration.

To the Applicant's knowledge, there has not been a description of means that would enable the isolation of a substantially pure population of adult cone photoreceptor cells. Particularly needed would be isolation means that would not promote the selection of a particular cone sub-population (S-cones vs. M/L-cones), i.e. that would introduce no selection bias, thereby giving a reliable in vitro model of the cone cell distribution in the tissue from which it derives. Particularly needed would also be isolation means that would be applicable to mammalian cone cells, and more particularly human cone cells. Most particularly needed would be isolation means that would be applicable to diseased adult cone cells.

Further to such isolation means, there also is a need for culture means that would enable to easily and reliably maintain isolated cone cells in in vitro culture.

### SUMMARY OF THE INVENTION

The present invention provides a process and kit for the production of a cone photoreceptor population of high purity. It thereby provides a high purity population of cone photoreceptor cells obtainable therefrom, and with means enabling the easy and reliable conducting of an in vitro culture of cone photoreceptor cells. The present invention also relates to the biological applications of such cone photoreceptor cell population and culture.

It more particularly relates to means enabling the isolation and culture of a cone cell population of high purity, wherein said means advantageously do not select a particular cone sub-population, thereby giving access to a cone culture that is reliably representative of the cone cell distribution that was initially present in the retinal tissue from which the cones have been isolated.

They also have the advantage of being applicable to, and efficient for adult cone photoreceptors. They further are applicable to mammalian cone cells, and more particularly to human cone cells.

The invention thereby gives access to genomics and proteomics, as well as to means for screening for compounds that have therapeutic and/or palliative and/or anti-degenerative effects on pathological or otherwise altered cone cells.

In the present application, reference is made to various scientific publications. These publications are listed in the “BIBLIOGRAPHIC REFERENCES” section which is located before the “CLAIMS” section.

### DETAILED DESCRIPTION

In order to characterize cone photoreceptors and screen trophic factor supporting their survival, the present invention provides a process that notably allows the specific isolation and culture of viable adult cone photoreceptors by “lectin-panning” using the peanut germ agglutinin lectin (PNA).

PNA specifically interacts with the cone domains of the interphotoreceptor matrix in the retinal tissue (Kivela and Tarkkanen, 1987; Uehara et al., 1997; Yan et al., 1995; Ahnelt and Kolt, 2000).

But, PNA was also reported to bind differently to the different populations of cone photoreceptors (Ebrey and Koutalos, 2001).

The present invention demonstrates that PNA can nevertheless be successfully used to mediate isolation of cone photoreceptor cells from an adult mammalian retinal tissue, and that PNA binding can still be operative and selective even though the tissue to which it is applied has undergone extracellular matrix dissociation.

It further shows that PNA can mediate said isolation without introducing a selection bias in the cone sub-populations, i.e. without promoting the adherence of a particular cone sub-population to the detriment of another particular cone sub-population.

It is indeed herein shown that PNA lectin-panning can select both S- and L/M-cone photoreceptors, and is efficient even though the retinal cells have undergone extracellular matrix lysis for cell dissociation.

The isolation means of the invention have the further advantages of being applicable to:
adult mammalian cone cells, and more particularly human cone cells, and to
healthy as well as pathological or otherwise altered cone cells.

The present invention further provides means enabling the in vitro culture of such isolated cone cells, demonstrating that retinal Müller glial cells can release diffusible factors that promote both adult cone photoreceptor survival and development in vitro.

The isolation and culture means of the invention thus give access to previously unmet industrial needs, notably for screening for compounds capable of showing therapeutic and/or palliative and/or anti-degenerative effects on adult diseased cone cells.

The present invention thus relates to a process for the in vitro production of a population of cone photoreceptors by isolation from a retinal tissue with a cone purity of at least 80%, typically of about 90%, said process promoting no selection of the S-cone sub-population vs. the L/M-cone sub-population.

Said process comprises:
optionally, chopping the retinal tissue into small fragments, preferably under conditions non deleterious to retinal cell biology, i.e. under conditions that do not substantially impair the growth potential of the retinal cells, for example in cold CO2-independent medium,
dissociating the extracellular matrix of the retinal tissue or retinal tissue fragments enzymatically and/or mechanically and/or chemically, so as to dissociate the retinal cells from each other without substantially altering their cellular integrity, i.e. without lysing the retinal cells,
optionally, suspending the dissociated cells in a solution,
placing said dissociated cells in contact with PNA (peanut germ agglutinin) under conditions enabling cell-PNA binding,
recovering the PNA-bound cells, and optionally in vitro culturing the PNA-bound cells,
   whereby said population of photoreceptor cones is obtained.

Said PNA is a commercially available and widely distributed product; for example, it is available from Sigma (reference: L0881)

Said purity percentage is assessed by counting the number of cells that are arrestin-positive, and relating it to the total number of cells that are observed under transmitted light.

To detect arrestin-positive cells, the skilled person can use the RT-PCR technique to detect cone arrestin mRNA (cf. example below, section “Material and Methods”, sub-section “single-cell RT-PCR”). Alternatively, an anti-arrestin antibody selective for cone arrestin protein can be used, e.g. an antibody to the QKAVEAEGDEGS epitope (cf. example below, section “Material and Methods”, sub-section “Histology”).

As illustrated in the Example below, a purity percentage of 92.57±2.12% assessed by anti-arrestin antibody (human anti-cone arrestin antibody; hCAR) was evaluated to be 92.06±4.82% by RT-PCR on arrestin mRNA.

The cell population obtained by the present invention comprises a majority of cone cells (namely at least 80%). There may nevertheless remain some “contaminant” cells, which originate from the retinal tissue on which the cell isolation has been performed, but which are not cone cells. These “contaminant” cells typically are:
rod bipolar cells, which typically may account for 4 to 6% of DAPI-stained nuclei, and also
very few glial cells, which were identified by their large DAPI-stained cell bodies.

These observations indicated that PNA-panning give rise to a high degree of purification of cone photoreceptors in culture.

Advantageously, notably compared to prior art techniques, as well as in the perspective of the possible uses that it offers, said retinal tissue can be an adult tissue. It may be a mammalian retinal tissue (primate or non-primate retinal tissue), e.g. a human retinal tissue, or an animal but non-human retinal tissue such as pig, rat, mouse retinal tissue.

Said retinal tissue can be a healthy tissue, as well as and advantageously a pathologic tissue, e.g. a tissue afflicted by a photoreceptor degeneration (cone and/or rod dysfunction).

In the process of the invention, said retinal tissue can therefore be a pathologic retinal tissue that undergoes or has undergone photoreceptor degeneration. This is a particularly advantageous aspect of the invention, as it enables methods for screening for compounds capable of showing a protective and/or anti-degenerative effect on cone cells, as well as genomics and proteomics applications, e.g. identifying genes and proteins that are specific or characteristic of the cone cells compared to the other retinal cells, or genes and proteins that are specific or characteristic of pathological cone cells compared to healthy cone cells (and vice versa).

Said pathologic retinal tissue can originate from an individual afflicted with an inherited or acquired disease involving photoreceptor degeneration, such as retinitis pigmentosa and age macular degeneration, or other maculopathies.

Said individual can advantageously be a mammal, such as a human or a non-human animal (e.g. pig, rat, mouse).

Dissociation of extracellular matrix of the retinal tissue or retinal tissue fragments can be achieved by any means available to the skilled person, as long as it allows for retinal cell dissociation without leading to cone cell lysis.

An enzyme, such as a proteolytic enzyme, can advantageously be used to that effect. Examples of such proteolytic enzymes notably include papain and trypsin. Papain is e.g. available from Worthington, and trypsin from Sigma.

The skilled person can adjust the dissociation conditions, such as temperature, enzyme concentration, and dissociation duration, as a function of the enzyme used and of the retinal tissue to which it is applied. Illustrative conditions are mentioned in the Example below. The skilled person may also use an enzyme activator, such as cysteine, if desired.

Dissociation can alternatively or additionally be performed mechanically and/or chemically. According to a particular aspect of the invention, dissociation is performed enzymatically and mechanically (see e.g. Example below, “Material and Methods” section, “Cell cultures” sub-section).

Before placing said dissociated cells in contact with PNA, the process of the invention can additionally comprise the step of saturating those sites which may introduce an aspecific lectin binding. Such a saturation may be achieved by any appropriate means available to the skilled person, e.g. by placing the dissociated cells obtained by disruption of said retinal tissue in contact with BSA.

According to an advantageous aspect of the invention, said PNA can be bound to a solid support, e.g. via anti-PNA antibody, such as the anti-PNA IgG available from Sigma under reference A4404.

Any solid support appropriate to cell binding can be used. It can e.g. be a cell culture plate, a glass coverslip, magnetic beads coupled with antibody anti-PNA.

Said placing in contact of the dissociated retinal cells with PNA can be achieved by any appropriate means that are appropriate to the binding of a lectin to a cell, such as by panning, MACS (magnetic activated cell sorting), or FACS (fluorescent activated cell sorting technique).

Once a cone population of a purity in cone cells as high as at least 80% has been produced, there comes the problem of maintaining such a population alive for a sufficiently long period of time, e.g. more than a week, such that they can be used in applications wherein cells shall preferably be living, such as screening protocols.

The present invention addresses this problem, and thereby discloses a fully comprehensive method for producing cone photoreceptors in vitro. It thus enables to perform automated screening procedures for e.g. the identification of therapeutically useful compounds.

A further and complementary aspect of the invention thus lies in the culture of the isolated cone photoreceptors.

The present invention notably demonstrates that Müller glial cells synthesize and extracellularly release trophic factors which promote the survival and development of cone cells.

The present invention hence provides a conditioned medium that is appropriate for in vitro cone cultures. The cells used to condition this medium are Müller glial cells.

Müller glial cells are available by e.g. purification from retinal suspensions, following the procedure described by Guidry (1996) [see Example below, “Material and Methods” section, “Müller glial cell conditioned medium” sub-section].

The “pre-conditioned” culture medium is selected as being suitable for cone survival, and may for example be NBA™ (Neurobasal Medium, available from Invitrogen), optionally supplemented by B27 and glutamine (cf. sub-section entitled “Müller glial cell conditioned medium” in Example Section “Material and Methods” below), or a DMEM/F12 medium (available e.g. from Invitrogen), or Ames (available e.g. from Sigma).

Once they have been purified, the Müller glial cells may first be in vitro cultured on a culture medium that is favourable to their own growth, such as e.g. DMEM-10% FCS, and then placed on the “pre-conditioned” medium, e.g. by substituting the first pro-growth culture medium by the pre-conditioned medium.

The process of the invention hence advantageously comprises in vitro culturing the PNA-bound cells on a conditioned culture medium obtainable by collection of the culture medium after in vitro culture of Müller glial cells.

The present invention also relates to any population of cone photoreceptors that is obtainable by the process of the invention. Such a population has a purity of at least 80% in cone cells, typically of 85-95%, e.g. about or higher than 90%.

As apparent from the description of the production process, said cone cells can advantageously be adult cone cells.

They can be mammalian (primate or non-primate) cone cells, e.g. human cone cells, or animal but non-human cone cells such as pig, rat, mouse cone cells.

As above-mentioned, said cone cells can derive from a healthy retinal tissue, as well as and advantageously from a pathologic retinal tissue afflicted by a photoreceptor degeneration (cone and/or rod dysfunction).

Said cone cells can thus be healthy cone cells, as well as and advantageously pathologic cone cells afflicted by a cone photoreceptor degeneration, such as those induced by retinitis pigmentosa, age macular degeneration, or any other maculopathy.

As previously mentioned, the process of the invention has the advantage of introducing no selection bias in the cone isolation: S-cone and L/M-cone photoreceptors are equally isolated.

If the retinal tissue to which the process of the invention is applied is a mammalian tissue, the population of cone photoreceptors will hence comprise both S-cone and L/M-cone photoreceptors.

The present invention also relates to any use of a culture medium obtainable by collection of the culture medium after in vitro culture of Müller glial cells for the in vitro culture of isolated cone cells.

It more particularly relates to a process for the production of a culture medium that is adapted to the in vitro culture of a population of cone photoreceptors, such as the population according to the invention. Said process comprises conditioning a culture medium that is suitable for cone survival with Müller glial cells, e.g. by culturing Müller glial cells onto a culture medium that is suitable for cone survival, and collecting the resulting culture medium, this collected culture medium being a conditioned culture medium adapted to the in vitro culture of a population of cone photoreceptors, such as a high purity population of cones according to the invention.

The pre-conditioned culture medium that is selected as being favourable to cone survival and that is intended to receive said Müller glial cells for culture is NBA™ culture medium, optionally supplemented by B27 and glutamine, or DMEM/F12, or Ames.

The conditioned medium is typically collected at day 1 or 2 of the Müller glial cell culture on pre-conditioned medium.

The present invention also relates to a cell culture which comprises:
a population of cone photoreceptors according to the invention, and
a conditioned culture medium obtainable by the process for the production of a culture medium of the invention.

The present invention also encompasses a multi-well culture plate, comprising in at least one of its wells, a population of cone photoreceptors according to the invention, and optionally a conditioned culture medium obtainable by the process for the production of a culture medium of the invention.

Are also encompassed by the present invention those kits that enable the implementation of the invention.

The present invention therefore encompasses a kit for the in vitro production of a population of cone photoreceptors by isolation from a retina tissue with a cone purity of at least 80%, typically of about 90%, said kit comprising:
a proteolytic enzyme, and
PNA, optionally bound onto a solid support,
and optionally, a culture medium intended for the in vitro culture of said cone cells, this culture medium being obtainable by collection of the culture medium after in vitro culture of Müller glial cells.

The kit may comprise either the culture medium resulting from collection of the culture medium after in vitro Müller glial cells (=the conditioned medium), or the Müller glial cells themselves and a (still uncultured yet) culture medium (=the pre-conditioned medium) which is intended to be first seeded by Müller glial cells, and then collected as resulting (conditioned) culture medium.

Said proteolytic enzyme may be any enzyme that allows the dissociation of the extracellular matrix of a retinal tissue or retinal tissue fragments, so as to dissociate the retinal cells from each other without lysing the retinal cells. Examples of such enzymes notably comprise papain, trypsin.

The present invention hence enables the isolation and long-lasting culture of populations of cone photoreceptors that reliably reflect the retinal tissue from which they have been isolated.

It therefore enables to screen for compounds capable of showing a protective and/or anti-degenerative effect on cone photoreceptors.

The present invention hence also relates to a screening method which comprises:
providing a population of cone photoreceptors that undergoes or has undergone cone degeneration, using the process according to the invention (i.e. from a retinal tissue afflicted by a photoreceptor degeneration such as retinitis pigmentosa, age macular degeneration or another maculopathy), said population being optionally seeded in a well of a multi-well plate,
placing said population of cone photoreceptors in contact with a candidate compound,
assessing whether said candidate compound induces a protective and/or anti-degenerative effect on said population of cone photoreceptors, e.g. by assessing whether it promotes cone cell viability or reduces cone cell mortality.

Candidate compounds include EGF, FGF2, and RDCVF1 and RDCVF2 (Rod-Derived Cone Viability Factor; see WO 02/081513).

Screening can be achieved e.g. by a Live/Dead assay (count of living and dead cells; e.g. using the Live/Dead assay kit from Molecular probes, Eugene, USA), by total cell count (DAPI staining), cone cell count (ELISA), and/or by morphometric determination. Said population of cone photoreceptors can be a population of animal but non-human animal cone photoreceptors, such as pig, rat or mouse cone photoreceptors.

For validation studies, a population of human cone photoreceptors can advantageously be used.

The present invention also relates to the use of a population of cone cells according to the invention in genomics and/or proteomics analysis processes, e.g. to identify a gene/protein that is specific or characteristic of the cones cells compared to the other retinal cells, to identify a gene/protein that is specific or characteristic of a pathological cone cell compared to a healthy cone cell (and vice versa).

The present application hence relates to any use of PNA for the isolation of cone photoreceptors, and to a culture medium enabling the survival of such isolated cones, said culture medium containing trophic factors that are extracellularly released by cultured Müller glial cells.

### BRIEF DESCRIPTION OF THE FIGURES

In the examples below, reference is made to the following figures:

FIG. 1. Photomicrographs of freshly lectin-panned cells. (A-B) Cone photoreceptor morphology observed under transmitted light with Nomarski optics immediately after the panning procedure. OS: outer segment IS: inner segment, CB: cell body, Ax.: axon and CP: cone pedicle. (C) Immunolabelling with hCAR, an antibody directed against the human cone-arrestin and (D), the nucleus marker DAPI. (E-F) Evidence for the presence of both cone populations in the lectin-panning preparation. Freshly panned cells were immunopositives for either S-opsin (F) or M/L-opsin (F). Opsins are shown in red and the nucleus marker DAPI in blue.

FIG. 2. Photomicrographs of pig retina immunofluorescence for cone photoreceptors markers. Pig retinal section observed under Nomarski optics (A) or epifluorescence after PNA labeling (B) or DAPI staining of the nuclei (C). (D-L) are focuses of the outer retina after labeling with PNA (D, G, J, green), hCAR (E,), L/M-opsin (H) or S-opsin (K) (red). (F-L) correspond to merged images of PNA, opsins and nucleus stainings. Note for instance that cone photoreceptors stained with PNA are also immunopositives for hCAR or cone-opsins, indicating that PNA-lectin equally labels both S-wavelength and M/L-wavelength cone photoreceptors in the pig retina.

FIGS. 3A-3D. Purity assessment of the lectin-panned cells. After 2 days in culture, isolated cells were counted under transmitted light (FIG. 3C) or under epifluorescence illumination with the nucleus marker DAPI (FIG. 3A) and the human anti-cone arrestin antibody (hCAR) (FIG. 3B). Differential counting revealed a 92.57±2.12% purity when comparing hCAR and phase contrast while comparison with DAPI is only 80.2±2.32% (FIG. 3D). (*) indicates picnotic cells that are discarded for counting in phase contrast.

FIG. 4. Analysis of single-cell RT-PCR. A 100 bp DNA standard marker ladder is shown on lanes M. Lanes 1-3 were negative controls: PCR buffers, intra-cellular pipette buffer and extra-cellular buffer, respectively. Lanes 4 to 10 show a 291 bp amplified DNA product corresponding to C-arrestin mRNA from a single cell. DNA products were resolved in a 1.2% agarose gel and visualized with ethidium bromide.

FIGS. 5A-5E.

FIG. 5E. Glial influence on cone photoreceptors survival. ANOVA of the number of hCAR-positive cells at different time in culture showed a significant “Day” effect [F( 1/12)=63.62, p<0.001] that was due to a 64% decrease in the total number of labeled cone photoreceptors at day 7 compared to day 4 of culture. ANOVA also showed a significant effect of the “Culture Medium” [F( 1/12)=8.94, p<0.05] that reflected a better survival in RMG CM compared to the control medium at any time in culture. (*) Different from the control condition at the same day (p<0.001); (**) Different from the control condition at Day 4 (p<0.01); (#) Different from Day 4 (p<0.05).

FIGS. 5A-5C. Glial influence on neuritis outgrowth. (A-B) Control medium and (C-D) Glia-conditioned medium.

FIGS. 6A-B. Voltage-activated conductances in cone photoreceptors recorded either in situ or after the lectin-panning-mediated purification.

FIG. 6B. I-V relationships of cells voltage-clamped at −70 mV and submitted to voltage steps of 10 mV increments, ranging from −120 to +50 mV.

FIG. 6A. Sample traces showing two representative cells recorded either in situ or in vitro, following the dissociation. Note for instance the highly homogenous whole cell conductances of the two conditions.

FIG. 7A. Sus scrofa arrestin-C (ARR3 gene) nucleotide sequence (EMBL accession number AJ564496; SEQ ID NO:5; encoded protein SEQ ID NO:6); and FIG. 7B. Sus scrofa S-antigen mRNA (accession number S82664; SEQ ID NO:7; encoded protein SEQ ID NO:8).

FIGS. 8A-C. Lectin-panning-mediated purification of human cone photoreceptors.

FIG. 8A. Photomicrograph of freshly prepared lectin-panned human cells, showing the classical morphology for human cone photoreceptors, observed under transmitted light with Nomarski optics immediately after the panning procedure.

FIG. 8B. Immunolabelling using hCAR, an antibody directed against the human cone-arrestin.

FIG. 8C. Quantification of the cone photoreceptors immunolabelled with an anti-(red/green opsin) antibody (R/V), or with an anti-(cone-arrestin) antibody (arrestine).

The following examples are offered by way of illustration and are not intended to limit the invention in any manner.

### EXAMPLES

### Material and Methods

Cell Cultures

Cell suspensions were prepared as described previously (Gaudin et al., 1996, Picaud et al., 1998a). Briefly, adult pig eyes were obtained from the local slaughterhouse. After a short alcohol treatment, the cornea, lens and vitreous humor were removed from the eyecup. The retinal tissue was detached from the pigmented epithelia and the underlying sclera. The retina was chopped into small fragments in cold CO₂-independent medium (Invitrogen). Retinal pieces were incubated for 20 min at 37° C. in a papain solution (1 U/μl; Worthington) previously activated by L-cystein (Sigma). The enzymatic reaction was stopped by adding 1 ml of neurobasal medium (NBA; Invitrogen) supplemented with 2% FCS (Invitrogen) and tissue aggregates were eliminated by adding DNase I (30 μl; Sigma) to the solution. The tissue was then gently shaken and a first supernatant was taken off after the pellet was decanted. The tissue was then gently triturated with a fire polished Pasteur pipette and different supernatants were collected until complete dissociation of the retinal tissue. The cell suspension was then centrifuged at 800 rpm for 5 minutes and cells resuspended in NBA (Invitrogen) supplemented with B27 (1:50, Invitrogen) and glutamine (1:100, Invitrogen) (NBA⁺).

For cell panning, glass coverslips were placed into Petri dishes (60 mm; Corning) and incubated for 2 hours at 37° C. with a goat anti-rabbit IgG directed against the PNA lectin (1:100; Sigma; A4404) diluted in 2 ml of Tris-HCl buffer (50 mM; pH 9.5). After 3 washes with warm PBS, coverslips were incubated in the Tris-HCl buffer containing the PNA lectin (1:40; Sigma). After 2 hours at 37° C., coverslips were again washed with PBS and transferred into 2 ml of D-PBS (Gibco) supplemented with BSA (0.2%; Fraction V; Sigma). The retinal suspension obtained was subsequently added onto the panned coverslips at a density of 4.10⁵ cells/cm² in 24-wells culture plates and incubated for 15 min while gently swirling the plates every 5 minutes. Wells were then washed 5 times with NBA in order to remove non adherent cells. Purified cells were finally incubated with either NBA⁺ or conditioned medium obtained from pure retinal Müller glial cell cultures (RMG CM) and maintained in vitro until patch-clamp recordings or immunohistochemistry while refreshing mediums every two days.

Müller Glial Cell Conditioned Medium

Conditioned medium (CM) were obtained from purified pig retinal Müller glial cells (RMG) cultured in NBA⁺. Müller cells were isolated following the procedure described by Guidry (1996) from cell suspensions prepared as described above. Briefly, the total retinal cell suspension was laid on a 10 ml continuous density gradient composed of 0-50% Percoll in normal saline and centrifuged for 5 minutes at 1700 rpm. The band located at the middle of the tube, containing the partly purified Müller glial cells, was taken off and submitted to a mild centrifugation (800 rpm for 5 minutes) in DMEM medium (Invitrogen) supplemented with 10% FCS in order to remove the Percoll. The pellet was resuspended in 1 ml DMEM-10% FCS and again centrifuged at 1700 rpm on a second Percoll gradient. The medium band was collected and the Percoll washed out by mild centrifugation.

The purified Müller glial cells were finally seeded at a density of 0.4×10⁵ cells/cm² in 6-well culture plates previously coated with poly-D-Lysine (1:100; Sigma) and laminine (1:200; Sigma) and cultured in DMEM-10% FCS.

24 hours later, cells were washed twice with DMEM-10% FCS in order to remove the remaining Percoll and allowed to grow. When the culture reached half-confluence, the NBA⁺ medium was substituted to the DMEM-10% FCS medium.

The RMG CM was collected every 2 days and immediately added to the lectin-panned cells.

Histology

Stainings were achieved as described previously (Gaudin et al., 1997; Picaud et al., 1998a,b). Briefly, isolated pig retinae were fixed in 4% paraformaldehyde (PAF) prepared in 0.1 M PBS (pH 7) for 1 h and cryoprotected by immersion in sucrose gradients. Retinal sections (8 μm) obtained on a cryostat were permeabilized with 0.1% Triton-X100 for 5 min. Cultured cells were similarly immersion fixed with 4% PAF for 15 min and permeabilized with 0.1% Triton-X100 for 5 min. In order to prevent unspecific labeling, cultures and retinal sections were incubated 1 h in a blocking buffer containing 10% goat serum and 2% bovine serum albumin in 0.01 M PBS.

Staining was achieved with PNA coupled to Alexa 488 (1:40-1:100, L-21409, Molecular Probes), a mouse monoclonal antibody directed against the PKCα (1:100, Sigma), a rabbit polyclonal antibody directed against the human cone arrestin (hCAR, 1:20.000-1:100.000, epitope=QKAVEAEGDEGS), a rabbit polyclonal to human S-cone opsin (AB5407; 1:10.000-1:20.000) and a rabbit polyclonal to human M/L-cone opsin (AB5405; 1:2000-1:5000), both commercially available from Chemicon.

All antibodies were diluted in the blocking buffer solution. For the immunolabeling, cells or retinal sections were incubated with the primary antibodies for 3 h at room temperature, rinsed several times with PBS, and incubated at 37° C. for 1 h with anti-rabbit or anti-mouse IgGs coupled to Alexa-594 (red emission) or Alexa-488 (green emission) (Molecular Probes). The Alexa 488-coupled PNA and the nucleus marker DAPI (1:200, Sigma) were incubated as secondary antibodies.

Cell Counting and Statistical Analysis

All experiments were realized in triplicates and cell counting was performed on 3 coverslips for each experiment. For each coverslip, cells were counted under the 40× objective in 30 adjacent areas along the diagonals. Results are expressed as ratio of number of hCAR-labeled cells versus number of DAPI-stained nuclei and/or Nomarski counting. Fluorescent labeling was observed using a Nikon Optiphot 2 Microscope under epifluorescence illumination. All images were acquired with a CCD color Cool-Snap FX camera and analyzed with the Metaview software (Roper Scientific Inc.).

The effect of culture mediums on cone photoreceptors survival was assessed by analysis of variance (ANOVA) followed, when appropriate, by 2×2 comparisons based on the Newmam-Keuls test (Winer, 1971). All analyses were conducted on data means of 3 coverslips after counting of hCAR-labeled cells under the 20× objective in 30 areas.

Patch Clamp Recordings

Cone photoreceptors were recorded with the patch clamp technique in the whole cell mode either freshly after dissociation or following 2 to 5 days in culture. Recording pipettes were pulled from thin-walled borosilicate glass (TW150F, World Precision Instruments) using a Brown and Flaming-type puller (P-87, Sutter Instruments). Cells were voltage clamped using an RK400 amplifier (Biologic). Data were acquired and analyzed using the Patchit and Tack software packages, respectively (Grant and Werblin, 1994).

For in vitro recordings, the standard perfusing solution had the following composition (in mM): 135 NaCl, 5 KCl, 1 CaCl₂, 1 MgCl₂, 10 glucose and 5 HEPES, pH adjusted to 7.74 with NaOH and was delivered by a general gravity-driven perfusion system (˜2 ml/min) at room temperature. The pipette solution contained (in mM): 140 KCl, 1 MgCl₂, 0.5 EGTA, 5 ATP, and 4 HEPES (pH adjusted to 7.4 with KOH). All chemicals were obtained from Sigma.

For in situ recordings, the perfusing solution was an Ames medium (Sigma) continuously bubbled with carbogen to equilibrate pH.

Retinal Slice Preparation

Small square pieces (4×4 mm²) of fresh pig retina were dissected out and flat mounted, photoreceptor side up, onto filter paper squares of bigger dimension. The whole preparation were immerged in cold Ames medium and 100˜150 μm sections were proceeded with a razor blade maintained in a stereotaxic frame. The slices were then transferred into a perfusion chamber and the filter paper incrusted on grease tubes in order to expose the retinal slices horizontally. Cells were filled with the sulforhodamine-101 fluorescent dye (Sigma) during the recording and observed under epifluorescence illumination (red emission). Cone photoreceptors were then counter-stained with PNA coupled to Alexa 488 (green emission).

Cloning of Sus scrofa Arrestin

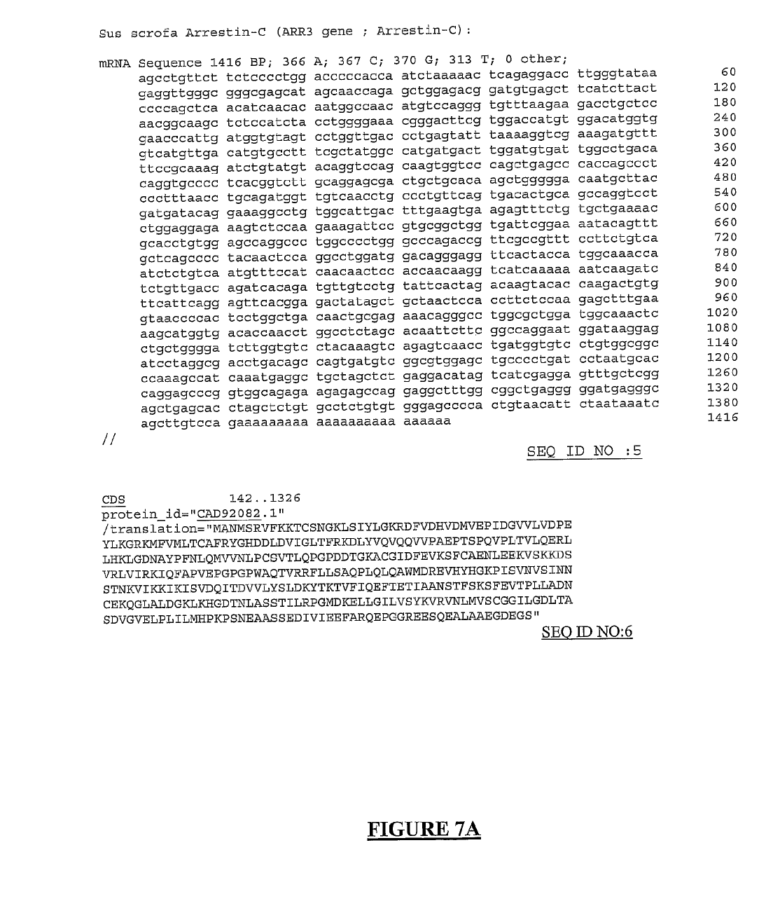

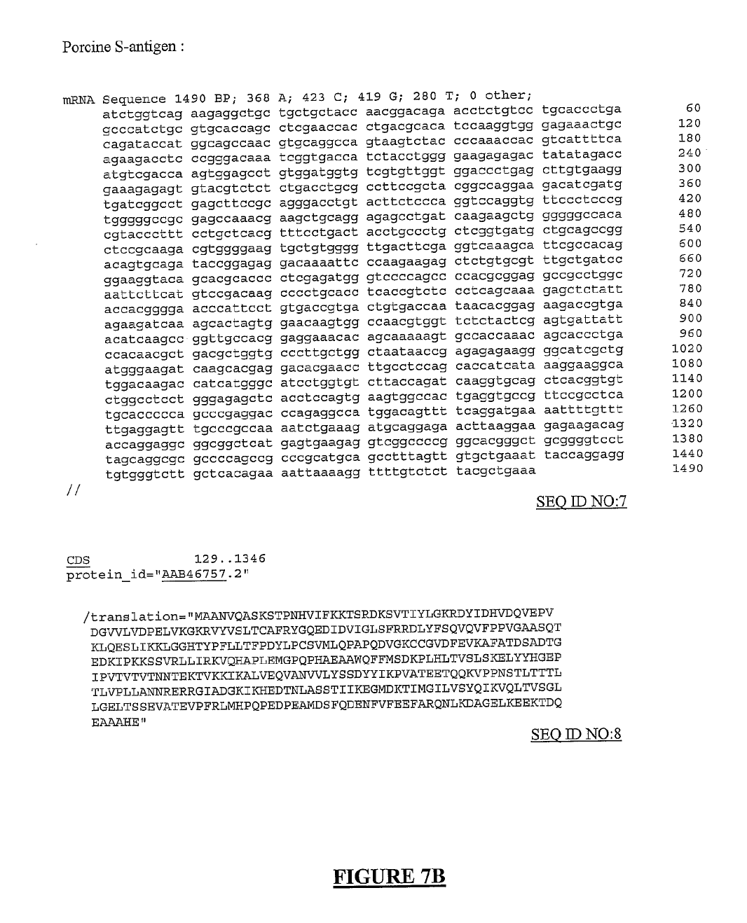

Total RNA from pig retina was prepared using Trizol reagent (Invitrogen) according to the protocol provided by the manufacturer. A 922 bp pig cone arrestin fragment was first cloned using oligonucleotides whose sequences were based upon the comparison of diverse mammals arrestin sequences. Then the complete pig arrestin 1416 nt coding sequence was obtained using Invitrogen 5′ and 3′ Race strategy: the cDNA sequence for Sus scrofa arrestin-C (ARR3 gene) was submitted to EMBL Nucleotide Sequence Database and delivered under the accession number AJ564496 (SEQ ID NO:5; FIG. 7A).

Single-Cell RT-PCR

Isolated cells were randomly picked up from coverslips using a patch clamp recording pipette filled with 8 μl of buffer solution (in mM): 140 KCl, 1 MgCl₂, 0.5 EGTA, 5 ATP and 4 HEPES (pH 7.4) and then pulled out into a thin wall PCR tube maintained in ice and containing 40 μl of the reaction mix SuperScript III One-Step RT-PCR system (Invitrogen) and couples of oligonucleotides. Tubes were placed at −80° C. until all the samples for one experiment have been collected. Tubes were allowed to slightly defreeze on ice and 2 μl of SuperScriptIII RT/Platinum Taq mix were added to each sample. The cDNA synthesis was achieved in a 30 min incubation at 50° C. followed by one cycle of denaturation at 94° C. for 2 min, 37 cycles of a PCR amplification (94° C. for 30″, 52° C. for 45″, 68° C. for 45″) and a final extension of one cycle at 68° C. for 5 min. A dilution of the original PCR was re-amplified using different couples of oligonucleotides (see below): 5 μl aliquot of the primary PCR was diluted into 495 μl TE buffer. Five μl of this diluted primary PCR product were used for a second Taq PCR (Invitrogen) cycle made of one cycle of denaturation at 94° C. for 2 min followed by 35 cycles (94° C. 30″, 55° C. 45″, 72° C. 45″) and one cycle at 72° C. for 5 min. Ten μl of the PCR product were analyzed on ethidium bromide staining agarose gel.

The sequences of oligonucleotides used for the first single-cell RT-PCR were as follow:

| | |
|---|---|
| +205 [GGGAAACGGGACTTCGTG] +222 | SEQ ID NO:1 |
| +593 [GCACAGAAACTCTTCACTTC] +574 | SEQ ID NO:2 |

The 389 bp fragment obtained from the primary RT-PCR was then amplified using the following oligonucleotides:

| | |
|---|---|
| +219 [CGTGGACCATGTGGACATG] +237 | SEQ ID NO:3 |
| +509 [AGGTTGACAACCATCTGCAG] +490 | SEQ ID NO:4 |

### Results

Lectin-Panning of Photoreceptors: Immunohistochemistry

We assessed whether the lectin PNA could promote the selective adherence of this cell population to a culture plate, whereas the cells had been subjected to enzymatic and mechanic treatments.

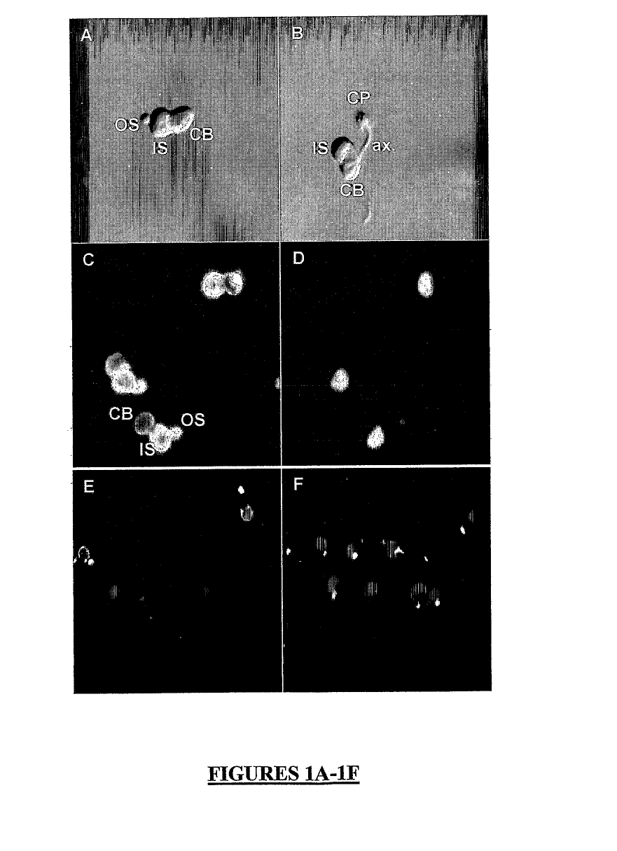

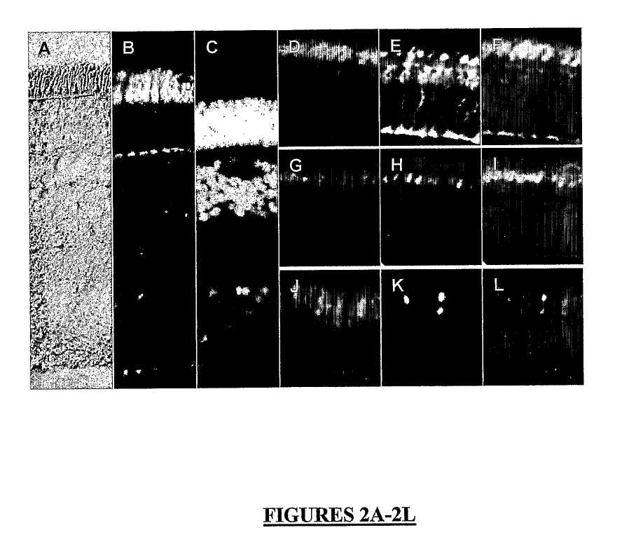

When cells were observed immediately after the lectin-panning procedure, most had the typical morphology of cone photoreceptors with a short outer segment, inner segment and cell body of similar sizes and a long axon ending by a large pedicle (FIG. 1A-B). When such cells were immunolabeled with the human anti-arrestin antibody (hCAR) that labels the entire cone cell cytoplasm in the pig retina (FIG. 2E), most cells appeared immunopositive (FIG. 1C). This observation indicated that cone photoreceptors can be purified by PNA-lectin-panning.

Since PNA was reported to bind differently to the different populations of cone photoreceptors (Ebrey and Koutalos, 2001), we verified that the lectin-panning procedure allowed the purification of all cone types. In the pig retina, PNA stained intensely the outer and inner segments of cone photoreceptors, more lightly their cell bodies located in the outer row of the outer nuclear layer (ONL), and heavily their axons and pedicles (FIG. 2B). Immunolabeling with the human cone arrestin antibody (hCAR) showed a complete colocalization with the PNA-lectin staining suggesting that both cone photoreceptor cell populations were stained by PNA in the pig retina. When sections were immunolabeled with the blue (Short wavelength, S-) or the red/green (Middle to Long wavelength, M/L-) cone opsin antibodies (FIG. 2G-L), both types of cone photoreceptors were stained by PNA. In the lectin-panning culture, both S- and L/M-opsin immunopositive cells were presents (FIG. 1E-F). These observations indicated that PNA lectin-panning can select both S- and L/M-cone photoreceptors.

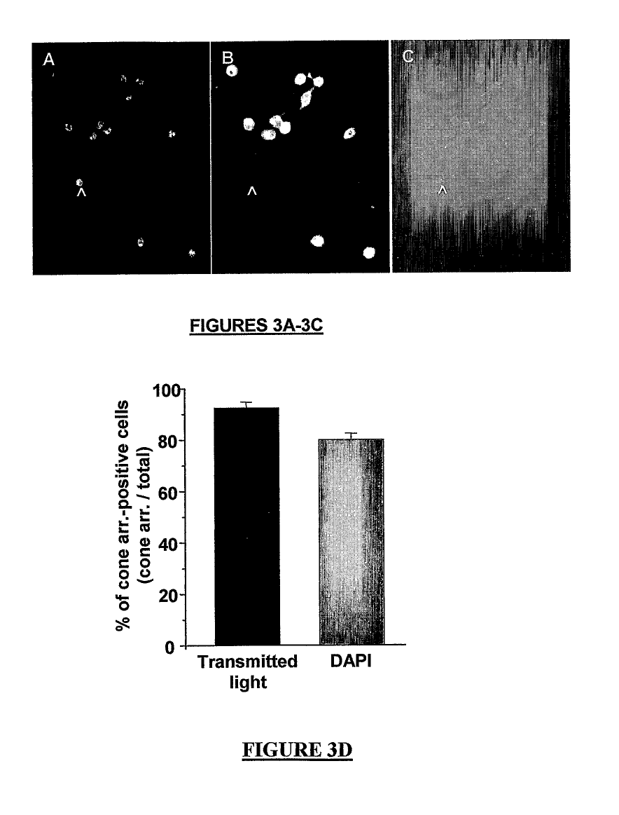

After two days in culture, lectin-panned cells lost their outer and inner segments and acquired an oval-shaped and medium-sized cell bodies with short neuritis (FIG. 3B). When these cultures were labeled with the cone arrestin antibody 24-48 h after the dissociation, all cells remained arrestin-immunopositives (FIG. 3B). Cone arrestin-positive cells accounted for 92.57±2.12% (s.e.m., n=9) of cells observed under transmitted light (FIG. 3C). To further assess the cone quantification, cultured cells were labeled with the nuclear dye DAPI (FIG. 1D). In this case, cone arrestin positive photoreceptors accounted for 80.2±2.32% (s.e.m., n=9) of DAPI-stained nuclei (FIG. 3A). The difference between the two counts may arise from DAPI-labeling of degenerated cells not taken into account in the first quantification. To identify contaminating cells, cultured cells were immunolabeled with the PKCα antibody which selectively labels rod bipolar cells (Karschin and Wässle, 1990). Our observations indicated that rod bipolar cells accounted for 5.58±0.05% (s.e.m., n=3) of DAPI-stained nuclei. Very few glial cells were detected (identification by their large DAPI-stained cell bodies). These observations indicated that lectin-panning give rise to a high degree of purification of cone photoreceptors in culture.

Lectin-Panning of Photoreceptors: Single-Cell RT-PCR

Adherent cells were picked up from 3 independent set of experiments. In the first experiment, 15 cells were collected immediately after the panning procedure. In a second experiment, 17 cells were picked up three days after the lectin-panning and in the last experiment, 15 cells were withdrawn five days after their adhesion.

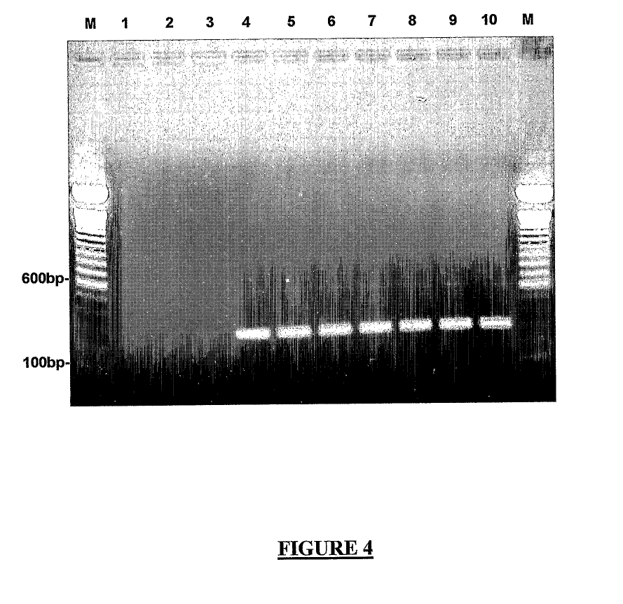

For each experiment, sources of contamination such as RT-PCR buffers, intra-cellular pipette buffer and perfusing solution (FIG. 4) were checked. In order to detect a possible genomic contamination, the Super-Script reverse transcriptase was omitted in three tubes for each experiment and replaced by Taq Platinum alone. The oligonucleotides used in this study were carefully designed and contained no less than seven mismatches with sequences located in the homologous regions of Sus scrofa S-antigen mRNA (accession number S82664; SEQ ID NO:7; cf. FIG. 7B). If any noticeable signal was absent in the different negative controls performed so far, an expected 291 bp product corresponding to the cone arrestin fragment was observed in most of the single-cell RT-PCR samples analysis. Positive signals observed in the first, second and third experiments were 12 out of 12, 13 out of 14 and 10 out of 12 respectively, corresponding to a 92.06±4.82% purity of the preparation (s.e.m., n=3).

### Glial Dependence of Cone Photoreceptor Survival

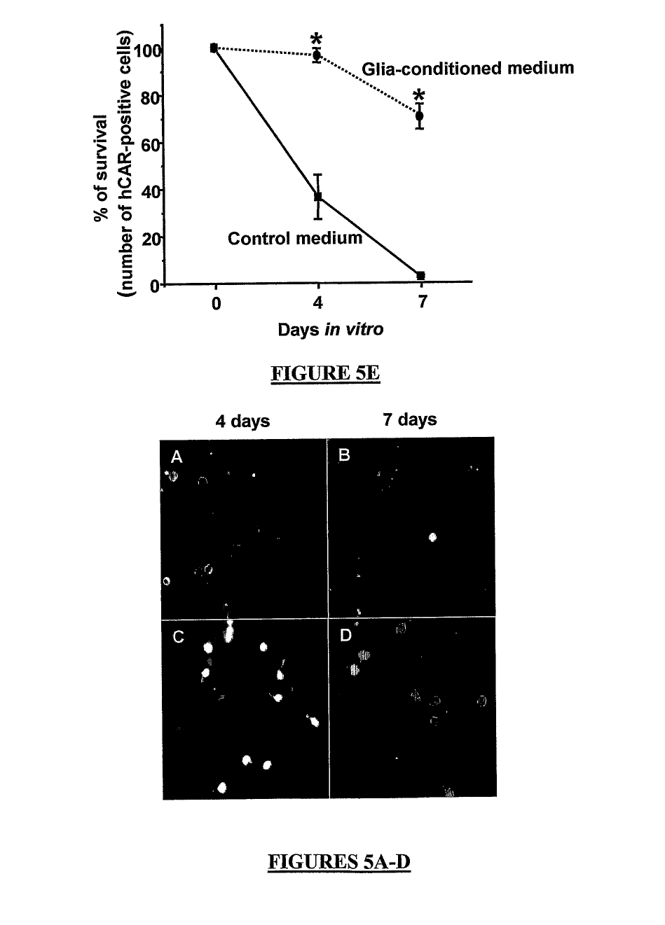

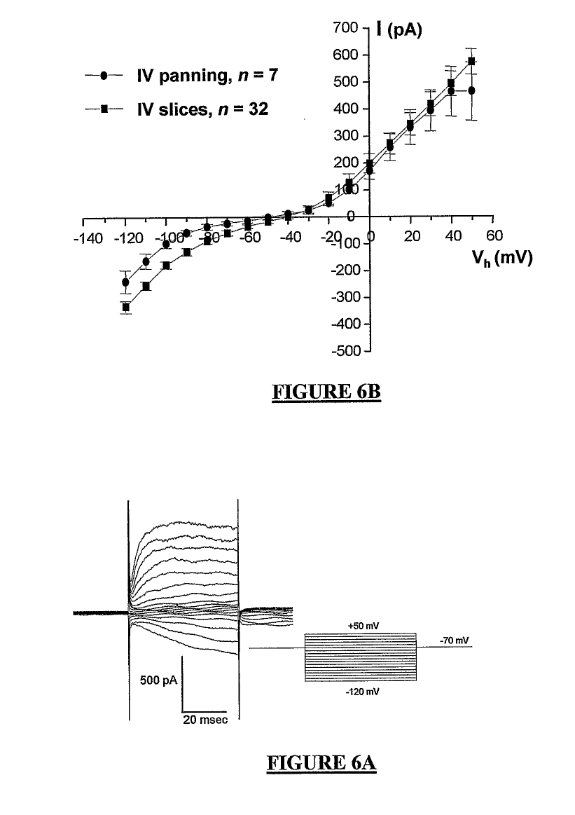

It was previously reported that Müller glial cells are required for the survival of adult pig photoreceptors isolated by vibratome sectioning of the retina (Picaud et al., 1998b). To assess whether cone photoreceptor rely on glial neurotrophic factors for their survival, cone photoreceptor cells isolated by PNA lectin-panning were cultured in the presence of glia conditioned medium (RMG CM, see material and methods). After 24 h in culture, no clear difference was observed in the number of cone photoreceptors between the RMG CM and control conditions. By contrast, after 4 days in culture a major difference was observed between the two mediums. While the number of hCAR-positive cells decreased by 69.41±8.97% in control conditions (s.e.m., n=12), it remained stable in the RMG CM, a 2.59 decrease (+9.44%; s.e.m., n=12) was only observed. After one week in vitro culture, the survival effect produced by the RMG CM was even more pronounced. The number of cone photoreceptors decreased by 96.48±0.65% (s.e.m., n=12) in control conditions whereas it only decreased by 31.10±8.13% in RMG CM (s.e.m., n=12) (FIG. 5E). These differences between the glia conditioned medium and control conditions at 4 days and at 1 week in culture were statistically significant at p<0.05. These results indicated that cultured glial cells can release molecules facilitating cone photoreceptor survival in culture.

When looking at morphological appearance of cells isolated by the PNA lectin-panning method no drastic differences appeared at 24 h between cells cultured in either control conditions or RMG CM. In both cases, cells had a rounded appearance with medium sized cell bodies showing sometimes short neuritis. However, after 4 days in culture, the number of cells developing neuritis was increased in RMG CM medium when compared to NBA⁺ medium (FIGS. 5A-D). These processes were still observed in cone photoreceptors cultured for 1 week in glia-conditioned media (FIG. 5 D). These results suggested that retinal Müller glial cells can release diffusible factors promoting both adult cone photoreceptor survival and development in vitro.

### Intrinsic Electrophysiological Homogeneity of Cultured Cells

To characterize the cone photoreceptor physiology, we used the whole cell configuration of the patch clamp technique to study the voltage-dependant activated currents in theses cells either in situ or after the panning procedure. FIG. 6A illustrates a sample trace of a typical cone recorded in a pig retinal slice. Cone photoreceptors showed a slowly-activating inward current at hyperpolarizing potentials (Vₕ=−70 mV, Iₘₐₓ=−58±9 pA; Vₕ=−120 mV, Iₘₐₓ=−329±19 pA, n=32) rising a peak amplitude within 50 msec (FIG. 6B). With a standard intracellular solution, the total current reversed near −40 mV. Outward currents activated at depolarizing potentials were of small amplitude (Vₕ=+50 mV, Iₘₐₓ=−575±47 pA, n=32), linear above −20 mV, and did not decayed with time. To assess their cell physiology in vitro, cone photoreceptors isolated by lectin-panning were recorded after the isolation. These cells expressed voltage-gated conductances similar to that of cone photoreceptors in situ excepted a slight increase in the whole cell conductances recorded at very hyperpolarized potentials. Recordings were obtained from 3 different cultures. These results confirmed that lectin-panned cells were viable and had physiological features of cone photoreceptors.

Lectin-Panning-Mediated Purification of Human Cone Photoreceptors

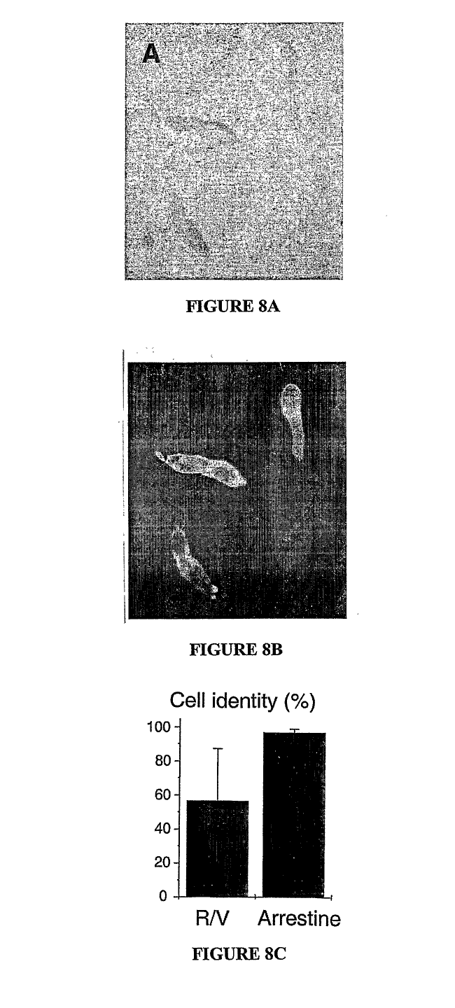

The PNA-lectin-panning-mediated process for the purification of cone photoreceptors was carried out independently on four post-mortem human retinas. When observing the purified cells under transmitted light with Nomarski optics immediately after the panning procedure, a large proportion of the cells exhibit the classical morphology for human cones (FIG. 8A). Immunolabelling using an antibody specific for human cone arrestin (FIG. 8B), confirms the identification of cone photoreceptors. A quantification of the thus immunolabelled cone photoreceptors on two retinas shows that the cones represent more than 95% of the cell population in the preparation. Depending on the retina, a variable proportion shows immunoreactivity for an antibody directed against red and green opsins (FIG. 8C).

These results indicate that the lectin-panning-mediated process for the purification of cone photoreceptors can be carried out with tissue from species other than pig, and in particular, with human retinal tissue.

### Discussion

We selected and assayed peanut agglutinin lectin (PNA) to design a procedure to purify selectively an adult cone cell population. The purification used PNA-coated cell culture plates. Incubation of a retinal cell suspension from the pig retina on this PNA-coated plates promoted the selective adherence of both S and L/M cone photoreceptors at a purity grade superior to 80%.

When Müller glial cell conditioned medium was incubated on lectin-purified cells, cone photoreceptor survival increased from 4 to 69% at 1 week. This study not only provides a new model to screen neuroprotective molecules for cone photoreceptors but it also open new possibilities for the characterization of cone photoreceptors in normal and pathological conditions.

Cone Photoreceptor Cell Sorting

The first attempts relating to cone isolation used chick embryo retina, which were in vitro cultured on low-density glia-free monolayers, and used to investigate retinal cell differentiation (Adler et al., 1984; see also review by Adler, 2000). For instance, retinal cells already postmitotic at embryonic day 5 (ED5) were shown to be almost 70% to differentiate as photoreceptors cells when cultured at ED6 whereas they were only 35% when isolated at ED8 (Adler and Hatlee, 1989).

Vibratome sectioning (Silverman and Hughes, 1989) and laser dissection (Salchow et al., 2001) of the retina has also been used in an attempt to purify photoreceptors. In culture, such isolated photoreceptors were found to survive for only a few days when prepared from young rat retina (Fontaine et al., 1998) or from embryonic human retina (Salchow et al., 2001). However, at the adult stage, such isolated photoreceptors required a glial feeder layer to survive (Picaud et al., 1998).

Finally, a progressive mechanic dissociation of enzymatically-treated retina also provided cultures enriched in photoreceptors (95%), but with a cone/photoreceptor ratio between 35-45% depending on the experimental conditions (Traverso et al., 2003).

The technique described in our study generated a 92% purity of adult fully differentiated cone photoreceptors. Cone cell identity was verified by their electrophysiological signature and by molecular markers, such as the cone arrestin or cone opsins, which were revealed at either gene expression with single RT-PCR or the protein with immunohistochemistry. The purity of the preparation appeared less important when cone cells were referred to the total number of DAPI-labeled cell nuclei rather than to the number of cells observed under transmitted light. This difference may rely on an overestimation of DAPI-labeled cell nuclei that are likely to include nuclear remnants of degenerated cells not identified as cells under transmitted light. PNA lectin-panning can therefore provide a high degree of purification of adult cone photoreceptors from retina.

The peanut agglutinin lectin (PNA) is typically considered a valuable marker of the extracellular matrix domain surrounding cone photoreceptor outer and inner segments (Ahnelt and Kolb, 2000). However, depending on species, the lectin labeling was not always identical depending on the cone spectral sensitivity thus revealing different compositions of their extracellular matrix domains. In ground squirrel, for instance, S-cones labeling is more intense than that of L-cones (Röhlich et al., 1989; Szél et al., 1993) while in fish and frog retina, PNA identifies L-cones selectively (Ishikawa et al., 1997). In the primate retina (Microcebus murinus), both S- and M/L-cones are decorated around their outer and inner segments, with an additional weak staining around their cell body and at their cone pedicle (Dkhissi-Benyahya et al., 2001). These observations are generally confirmed in most mammalian species like mouse and rats with intense labeling around cone outer/inner segments and at cone pedicles (Blanks and Johnson, 1983; Fei, 2003). In the pig retina, the labeling was similarly observed around both S- and M/L-cones (FIG. 2).

Cone photoreceptor labeling is not only observed in normal conditions but also in pathological states. Following rod photoreceptor loss and during their own secondary degeneration, cone cells were indeed identified and quantified by PNA labeling in the rd1 mouse retina of the living animal (Mohand Said et al., 1997) or following explant culture (Mohand-Said et al., 1998).

The present results demonstrate that PNA can nevertheless be successfully used to mediate isolation of cone photoreceptor cells from an adult mammalian retinal tissue, and that PNA binding can still be operative and selective even though the tissue to which it is applied has undergone extracellular matrix dissociation.

FACS technique using fluorescent PNA lectin is much likely to be very efficient in sorting cone photoreceptors.

Glial Neuroprotection of Cone Photoreceptors

In culture, the absence of glial cells did not prevent chick embryo cone photoreceptors to survive (Adler et al., 1984). By contrast, glial cells appeared very important for the survival of neonatal and adult mammalian rod photoreceptors (Kjavin and Reh, 1991; Hicks et al, 1994). The absolute glial dependence of photoreceptors was demonstrated when rods and cones isolated by vibratome sectioning the outer retina were found to survive only on a glial feeder layer (Picaud et al, 1998). This glial cell dependence of photoreceptors was further supported by the neuroprotective effect of neurotrophic factors like BDNF, GDNF, CNTF, that do not seem to have functional receptors on photoreceptors but instead on glial cells (Ugolini et al., 1995; Kirsch et al., 1998; Harada et al., 2003). Photoreceptor survival would indeed rely on Müller cell activation (Wahlin et al., 2000; Harada et al., 2003). Our results clearly establish that cone photoreceptors are dependent upon a diffusible trophic molecule released by retinal glial cells for their survival.

Retinal glial cells were reported to contain, and synthesize many growth factors and cytokines (Hicks et al., 1991; Mascarelli et al., 1991; La Vail et al., 1992; Li et al., 1995, 1997; Milam et al., 1997). The notion that glial cells synthesize trophic factors for retinal cells is consistent with the survival and outgrowth of retinal ganglion cells in the presence of a glia-conditioned medium (Garcia et al., 2002). It is also in agreement with the BDNF, GDNF and CNTF neuroprotection of photoreceptors that may require Müller cell activation (see above). Future studies should therefore focus on the isolation of such glia-derived trophic molecules.

### CONCLUSIONS

The present study provides a strategy to isolate cone photoreceptors that is very valuable to characterize cone photoreceptor at a genomic and proteomic level in both normal and pathological conditions. Already, its use in culture demonstrated the glial dependence of cone photoreceptors opening the search for glia-derived cone trophic factors.

### BIBLIOGRAPHIC REFERENCES

Adler R. A model of retinal cell differentiation in the chick embryo. Prog Retin Eye Res. 2000; 19:529-57.
Adler R, Hatlee M. Plasticity and differentiation of embryonic retinal cells after terminal mitosis. Science. 1989; 243:391-3.
Adler R, Lindsey J D, Elsner C L. Expression of cone-like properties by chick embryo neural retina cells in glial-free monolayer cultures. J. Cell Biol. 1984; 99:1173-8.
Ahnelt P K, Kolb H. The mammalian photoreceptor mosaic-adaptive design. Prog Retin Eye Res. 2000; 19:711-77.
Blanks J C, Johnson L V. Selective lectin binding of the developing mouse retina. J Comp Neurol. 1983; 221:31-41.
Chandler M J, Smith P J, Samuelson D A, MacKay E O. Photoreceptor density of the domestic pig retina. Vet Ophthalmol. 1999; 2:179-184.
Dkhissi-Benyahya O, Szel A, Degrip W J, Cooper H M. Short and mid-wavelength cone distribution in a nocturnal Strepsirrhine primate (Microcebus murinus). J Comp Neurol. 2001; 438:490-504.
Ebrey T, Koutalos Y. Vertebrate photoreceptors. Prog Retin Eye Res. 2001; 20:49-94.
Fei Y. Development of the cone photoreceptor mosaic in the mouse retina revealed by fluorescent cones in transgenic mice. Mol. Vis. 2003; 9:31-42.
Fintz A C, Audo I, Hicks D, Mohand-Said S, Leveillard T, Sahel J. Partial characterization of retina-derived cone neuroprotection in two culture models of photoreceptor degeneration. Invest Opthalmol Vis Sci. 2003; 44:818-25.
Fontaine V, Kinkl N, Sahel J, Dreyfus H, Hicks D. Survival of purified rat photoreceptors in vitro is stimulated directly by fibroblast growth factor-2. J. Neurosci. 1998; 18:9662-72.
Garcia M, Forster V, Hicks D, Vecino E. Effects of muller glia on cell survival and neuritogenesis in adult porcine retina in vitro. Invest Opthalmol Vis Sci. 2002; 43:3735-43.
Gaudin C, Forster V, Sahel J, Dreyfus H, Hicks D. Survival and regeneration of adult human and other mammalian photoreceptors in culture. Invest Opthalmol Vis Sci. 1996; 37:2258-68.
Geller A M, Sieving P A. Assessment of foveal cone photoreceptors in Stargardt's macular dystrophy using a small dot detection task. Vision Res. 1993 July; 33(11):1509-24.
Guidry C. Isolation and characterization of porcine Muller cells. Myofibroblastic dedifferentiation in culture. Invest Opthalmol Vis Sci. 1996; 37:740-52.
Harada C, Harada T, Quah H M, Maekawa F, Yoshida K, Ohno S, Wada K, Parada L F, Tanaka K. Potential role of glial cell line-derived neurotrophic factor receptors in Muller glial cells during light-induced retinal degeneration. Neuroscience. 2003; 122:229-35.
Hendrickson A, Hicks D. Distribution and density of medium- and short-wavelength selective cones in the domestic pig retina. Exp Eye Res. 2002; 74:435.
Hicks D, Forster V, Dreyfus H, Sahel J. Survival and regeneration of adult human photoreceptors in vitro. Brain Res. 1994; 643:302-5.
Ishikawa M, Hashimoto Y, Tonosaki A, Sakuragi S. Preference of peanut agglutinin labeling for long-wavelength-sensitive cone photoreceptors in the dace retina. Vision Res. 1997; 37:383-7.
Karschin A, Wassle H. Voltage- and transmitter-gated currents in isolated rod bipolar cells of rat retina. J Neurophysiol. 1990; 63:860-76.
Kirsch M, Schulz-Key S, Wiese A, Fuhrmann S, Hofmann H. Ciliary neurotrophic factor blocks rod photoreceptor differentiation from postmitotic precursor cells in vitro. Cell Tissue Res. 1998; 291:207-16.
Kivela T, Tarkkanen A. A lectin cytochemical study of glycoconjugates in the human retina. Cell Tissue Res. 1987; 249:277-88.
Kljavin I J, Reh T A. Muller cells are a preferred substrate for in vitro neurite extension by rod photoreceptor cells. J Neurosci. 1991; 11:2985-94.
LaVail M M, Unoki K, Yasumura D, Matthes M T, Yancopoulos G D, Steinberg R H. Multiple growth factors, cytokines, and neurotrophins rescue photoreceptors from the damaging effects of constant light. Proc Natl Acad Sci USA. 1992; 89:11249-53.
Li Z Y, Chang J H, Milam A H. Distribution of basic fibroblast growth factor in human retinas with retinitis pigmentosa. Exp Eye Res. 1997; 65:855-9.
Li Z Y, Chang J H, Milam A H. A gradient of basic fibroblast growth factor in rod photoreceptors in the normal human retina. Vis Neurosci. 1997; 14:671-9.
Li Z Y, Kljavin I J, Milam A H. Rod photoreceptor neurite sprouting in retinitis pigmentosa. J. Neurosci. 1995; 15:5429-38.
Mascarelli F, Tassin J, Courtois Y. Effect of FGFs on adult bovine Muller cells: proliferation, binding and internalization. Growth Factors. 1991; 4:81-95.
Milam A H, Li Z Y, Fariss R N. Histopathology of the human retina in retinitis pigmentosa. Prog Retin Eye Res. 1998; 17:175-205.
Mohand-Said S, Deudon-Combe A, Hicks D, Simonutti M, Forster V, Fintz A C, Leveillard T, Dreyfus H, Sahel J A. Normal retina releases a diffusible factor stimulating cone survival in the retinal degeneration mouse. Proc Natl Acad Sci U S A. 1998; 95:8357-62.
Mohand-Said S, Hicks D, Simonutti M, Tran-Minh D, Deudon-Combe A, Dreyfus H, Silverman M S, Ogilvie J M, Tenkova T, Sahel J. Photoreceptor transplants increase host cone survival in the retinal degeneration (rd) mouse. Ophthalmic Res. 1997; 29:290-7.
Picaud S, Hicks D, Forster V, Sahel J, Dreyfus H. Adult human retinal neurons in culture: Physiology of horizontal cells. Invest Opthalmol Vis Sci. 1998; 39:2637-48.
Picaud S, Pattnaik B, Hicks D, Forster V, Fontaine V, Sahel J, Dreyfus H. GABAA and GABAC receptors in adult porcine cones: evidence from a photoreceptor-glia co-culture model. J. Physiol. 1998; 513 (Pt 1):33-42.
Rohlich P, Szel A, Johnson L V, Hageman G S. Carbohydrate components recognized by the cone-specific monoclonal antibody CSA-1 and by peanut agglutinin are associated with red and green-sensitive cone photoreceptors. J Comp Neurol. 1989; 289:395-400.
Rohlich P, Szel A, Papermaster D S. Immunocytochemical reactivity of Xenopus laevis retinal rods and cones with several monoclonal antibodies to visual pigments. J Comp Neurol. 1989; 290:105-17.
Salchow D J, Trokel S L, Kjeldbye H, Dudley T, Gouras P. Isolation of human fetal cones. Curr Eye Res. 2001; 22:85-9.
Silverman M S, Hughes S E. Transplantation of photoreceptors to light-damaged retina. Invest Opthalmol Vis Sci. 1989; 30:1684-90.
Szel A, Rohlich P, Mieziewska K, Aguirre G, van Veen T. Spatial and temporal differences between the expression of short- and middle-wave sensitive cone pigments in the mouse retina: a developmental study. J Comp Neurol. 1993; 331:564-77.
Szel A, Rohlich P, Van Veen T. Short-wave sensitive cones in the rodent retinas. Exp Eye Res. 1993; 57:503-5.
Szel A, von Schantz M, Rohlich P, Farber D B, van Veen T. Difference in PNA label intensity between short- and middle-wavelength sensitive cones in the ground squirrel retina. Invest Opthalmol Vis Sci. 1993; 34:3641-5.
Traverso V, Kinkl N, Grimm L, Sahel J, Hicks D. Basic fibroblast and epidermal growth factors stimulate survival in adult porcine photoreceptor cell cultures. Invest Opthalmol Vis Sci. 2003; 44:4550-8.
Travis G H. Mechanisms of cell death in the inherited retinal degenerations. Am J Hum Genet. 1998; 62:503-8.
Uehara F, Yanagita T, Iwakiri N, Ohba N. Immunohistochemical localization of MUC1 glycoprotein in the retina. Jpn J. Opthalmol. 1997 May-June; 41(3):200-1.
Ugolini G, Cremisi F, Maffei L. TrkA, TrkB and p75 mRNA expression is developmentally regulated in the rat retina. Brain Res. 1995; 704:121-4.
Wahlin K J, Campochiaro P A, Zack D J, Adler R. Neurotrophic factors cause activation of intracellular signaling pathways in Muller cells and other cells of the inner retina, but not photoreceptors. Invest Opthalmol Vis Sci. 2000; 41:927-36.
Yan Q, Bumsted K, Hendrickson A. Differential peanut agglutinin lectin labeling for S and L/M cone matrix sheaths in adult primate retina. Exp Eye Res. 1995; 61:763-6.

1. Process for the in vitro production of a population of cone photoreceptors by isolation from a retinal tissue with a cone purity of at least 80%, typically of about 90%, said process promoting no selection of the S-cone sub-population vs. the L/M-cone sub-population, wherein said process comprises:
optionally, chopping the retinal tissue into small fragments,
dissociating the extracellular matrix of the retinal tissue or retinal tissue fragments enzymatically and/or mechanically and/or chemically, so as to dissociate the retinal cells from each other without lysing the retinal cells,
optionally, suspending the dissociated cells in a solution,
placing said dissociated cells in contact with PNA (peanut germ agglutinin) under conditions enabling cell-PNA binding,
recovering the PNA-bound cells, and optionally in vitro culturing the PNA-bound cells,
whereby said population of photoreceptor cones is obtained.

2. Process according to claim 1, characterized in that said retinal tissue is an adult tissue.

3. Process according to any one of the preceding claims, characterized in that said retinal tissue is a mammalian retinal tissue.

4. Process according to any one of claims 1-3, characterized in that said retinal tissue is a human retinal tissue.

5. Process according to any one of claims 1-3, characterized in that said retinal tissue is a animal but non-human retinal tissue, such as a pig, rat or mouse retinal tissue.

6. Process according to any one of the preceding claims, characterized in that said retinal tissue is a pathologic retinal tissue that undergoes or has undergone photoreceptor degeneration.

7. Process according to claim 6, characterized in that said pathologic retinal tissue originates from an individual afflicted with an inherited or acquired disease involving photoreceptor degeneration, said disease being selected from the group consisting of retinitis pigmentosa and age macular degeneration.

8. Process according to any one of the preceding claims, characterized in that said dissociation of the extracellular matrix of the retinal tissue or retinal tissue fragments comprises an enzymatic dissociation performed with a proteolytic enzyme.

9. Process according to claim 8, characterized in that said proteolytic enzyme is papain.

10. Process according to claim 8, characterized in that said proteolytic enzyme is trypsin.

11. Process according to any one of claims 8-10, characterized in that said dissociation further comprises a mechanical dissociation.

12. Process according to any one of the preceding claims, characterized in that said PNA is bound to a solid support.

13. Process according to any one of the preceding claims, characterized in that said placing in contact of the dissociated retinal cells with PNA is achieved by panning, MACS (magnetic activated cell sorting), or FACS (fluorescent activated cell sorting technique).

14. Process according to any one of the preceding claims, characterized in that it comprises in vitro culturing the PNA-bound cells on a conditioned medium obtainable by collection of the culture medium after in vitro culture of Müller glial cells.

15. Process according to claim 14, characterized in that said conditioned medium is obtainable by collection of the culture medium after in vitro culture of Müller glial cells on NBA™ (Neurobasal Medium), optionally supplemented by B27 and glutamine.

16. A population of cone photoreceptors obtainable by the process according to any one of claims 1 to 15, characterized in that it has a purity of at least 80% in cone cells.

17. A population of cone photoreceptors according to claim 16, characterized in that said cone cells are adult cone cells.

18. A population of cone photoreceptors according to any one of claims 16-17, characterized in that said cone cells are mammalian cone cells.

19. A population of cone photoreceptors according to any one of claims 16-18, characterized in that said cone cells are human cone cells.

20. A population of cone photoreceptors according to any one of claims 16-18, characterized in that said cone cells are animal but non-human cone cells, such as pig, rat or mouse cone cells.

21. A population of cone photoreceptors according to any one of claims 16-20, characterized in that it comprises both S-cone and L/M-cone photoreceptors.

22. Use of a conditioned culture medium obtainable by collection of the culture medium after in vitro culture of Müller glial cells, for the in vitro culture of cone cells.

23. Process for the production of a culture medium that is adapted to the in vitro culture of a population of cone photoreceptors, such as the population according to any one of claims 16-21, said process comprising conditioning a culture medium that is suitable for cone survival with Müller glial cells, by culturing Müller glial cells onto a culture medium that is suitable for cone survival, and collecting the resulting culture medium, this collected culture medium being is adapted to the in vitro culture of a population of cone photoreceptors, such as the one according to any one of claims 16-21.

24. Process for the production of a culture medium according to claim 23, characterized in that the pre-conditioned culture medium that is selected as being favourable to cone survival and that is intended to receive said Müller glial cells for culture is NBA™ culture medium, optionally supplemented by B27 and glutamine, or DMEM/F12 or Ames.

25. Cell culture which comprises:
a population of cone photoreceptors according to any one of claims 16-21, and
a culture medium obtainable by the process of claim 23 or 24.

26. Multi-well culture plate, comprising in at least one of its wells, a population of cone photoreceptors according to any one of claims 16-21, and optionally a culture medium obtainable by the process of claim 23 or 24.

27. Kit for the in vitro production of a population of cone photoreceptors by isolation from a retina tissue with a cone purity of at least 80%, typically of about 90%, said kit comprising:
a proteolytic enzyme, and
PNA, optionally bound on a solid support,
and optionally, a culture medium intended for the in vitro culture of said cone cells, this culture medium being obtainable by collection of the culture medium after in vitro culture of Müller glial cells.

28. Kit according to claim 27, characterized in that said proteolytic enzyme is papain or trypsin.

29. Method of screening for compounds capable of showing protective and/or anti-degenerative properties on cone photoreceptors, characterized in that it comprises:
providing a population of cone photoreceptors that undergoes or has undergone cone degeneration, using the process according to any one of claims 6-7, optionally placing this population in the wells of a multi-well culture plate,
placing said population of cone photoreceptors in contact with a candidate compound,
assessing whether said candidate compound induces a protective and/or anti-degenerative effect on said population of cone photoreceptors, e.g. by assessing whether it promotes cone cell viability or reduces cone cell mortality.

30. Method according to claim 29, characterized in that said population of cone photoreceptors is a population of animal but non-human animal cone photoreceptors, such as pig, rat or mouse cone photoreceptors.

31. Method according to claim 29, characterized in that said population of cone photoreceptors is a population of human cone photoreceptors.


SEQUENCE LISTING (8 sequences)

SEQ ID NO: 1 - DNA, 18 bp, Artificial Sequence
  Synthetic primer
gggaaacgggacttcgtg

SEQ ID NO: 2 - DNA, 20 bp, Artificial Sequence
  Synthetic primer
gcacagaaactcttcacttc

SEQ ID NO: 3 - DNA, 19 bp, Artificial Sequence
  Synthetic primer
cgtggaccatgtggacatg

SEQ ID NO: 4 - DNA, 20 bp, Artificial Sequence
  Synthetic primer
aggttgacaaccatctgcag

SEQ ID NO: 5 - DNA, 1416 bp, Sus scrofa
  CDS (142)..(1326)
agcctgttcttctcccctggacccccaccaatctaaaaactcagaggaccttgggtataagaggttgggcgggcgagcatagcaaccagagctggagacggatgtgagcttcatcttactccccagctcaacatcaacacaatggccaacatgtccagggtgtttaagaagacctgctccaacggcaagctctccatctacctggggaaacgggacttcgtggaccatgtggacatggtggaacccattgatggtgtagtcctggttgaccctgagtatttaaaaggtcgaaagatgtttgtcatgttgacatgtgcctttcgctatggccatgatgacttggatgtgattggcctgacattccgcaaagatctgtatgtacaggtccagcaagtggtcccagctgagcccaccagccctcaggtgcccctcacggtcttgcaggagcgactgctgcacaagctgggggacaatgcttacccctttaacctgcagatggttgtcaacctgccctgttcagtgacactgcagccaggtcctgatgatacaggaaaggcctgtggcattgactttgaagtgaagagtttctgtgctgaaaacctggaggagaaagtctccaagaaagattccgtgcggctggtgattcggaaaatacagtttgcacctgtggagccaggccctggcccctgggcccagaccgttcgccgtttccttctgtcagctcagcccctacaactccaggcctggatggacagggaggttcactaccatggcaaaccaatctctgtcaatgtttccatcaacaactccaccaacaaggtcatcaaaaaaatcaagatctctgttgaccagatcacagatgttgtcctgtattcactagacaagtacaccaagactgtgttcattcaggagttcacggagactatagctgctaactccaccttctccaagagctttgaagtaaccccactcctggctgacaactgcgagaaacagggcctggcgctggatggcaaactcaagcatggtgacaccaacctggcctctagcacaattcttcggccaggaatggataaggagctgctggggatcttggtgtcctacaaagtcagagtcaacctgatggtgtcctgtggcggcatcctaggcgacctgacagccagtgatgtcggcgtggagctgcccctgatcctaatgcacccaaagccatcaaatgaggctgctagctctgaggacatagtcatcgaggagtttgctcggcaggagcccggtggcagagaagagagccaggaggctttggcggctgagggggatgagggcagctgagcacctagctctgtgcctctgtgtgggagccccactgtaacattctaataaatcagcttgtccagaaaaaaaaaaaaaaaaaaaaaaaaa

SEQ ID NO: 6 - protein, 394 aa, Sus scrofa
MANMSRVFKKTCSNGKLSIYLGKRDFVDHVDMVEPIDGVVLVDPEYLKGRKMFVMLTCAFRYGHDDLDVIGLTFRKDLYVQVQQVVPAEPTSPQVPLTVLQERLLHKLGDNAYPFNLQMVVNLPCSVTLQPGPDDTGKACGIDFEVKSFCAENLEEKVSKKDSVRLVIRKIQFAPVEPGPGPWAQTVRRFLLSAQPLQLQAWMDREVHYHGKPISVNVSINNSTNKVIKKIKISVDQITDVVLYSLDKYTKTVFIQEFTETIAANSTFSKSFEVTPLLADNCEKQGLALDGKLKHGDTNLASSTILRPGMDKELLGILVSYKVRVNLMVSCGGILGDLTASDVGVELPLILMHPKPSNEAASSEDIVIEEFARQEPGGREESQEALAAEGDEGS

SEQ ID NO: 7 - DNA, 1490 bp, Sus scrofa
  CDS (129)..(1346)
atctggtcagaagaggctgctgctgctaccaacggacagaacctctgtcctgcaccctgagcccatctgcgtgcaccagcctcgaaccacctgacgcacatccaaggtgggagaaactgccagataccatggcagccaacgtgcaggccagtaagtctaccccaaaccacgtcattttcaagaagacctcccgggacaaatcggtgaccatctacctggggaagagagactatatagaccatgtcgaccaagtggagcctgtggatggtgtcgtgttggtggaccctgagcttgtgaagggaaagagagtgtacgtctctctgacctgcgccttccgctacggccaggaagacatcgatgtgatcggcctgagcttccgcagggacctgtacttctcccaggtccaggtgttccctcccgtgggggccgcgagccaaacgaagctgcaggagagcctgatcaagaagctggggggccacacgtaccctttcctgctcacgtttcctgactacctgccctgctcggtgatgctgcagccggctccgcaagacgtggggaagtgctgtggggttgacttcgaggtcaaagcattcgccacagacagtgcagataccggagaggacaaaattcccaagaagagctctgtgcgtttgctgatccggaaggtacagcacgcacccctcgagatgggtccccagccccacgcggaggccgcctggcaattcttcatgtccgacaagcccctgcacctcaccgtctccctcagcaaagagctctattaccacggggaacccattcctgtgaccgtgactgtgaccaataacacggagaagaccgtgaagaagatcaaagcactagtggaacaagtggccaacgtggttctctactcgagtgattattacatcaagccggttgccacggaggaaacacagcaaaaagtgccaccaaacagcaccctgaccacaacgctgacgctggtgcccttgctggctaataaccgagagagaaggggcatcgctgatgggaagatcaagcacgaggacacgaaccttgcctccagcaccatcataaaggaaggcatggacaagaccatcatgggcatcctggtgtcttaccagatcaaggtgcagctcacggtgtctggcctcctgggagagctcacctccagtgaagtggccactgaggtgccgttccgcctcatgcacccccagcccgaggacccagaggccatggacagttttcaggatgaaaattttgtttttgaggagtttgcccgccaaaatctgaaagatgcaggagaacttaaggaagagaagacagaccaggaggcggcggctcatgagtgaagaggtcggccccgggcacgggctgcggggtccttagcaggcgcgccccagccgcccgcatgcagcctttagttgtgctgaaattaccaggaggtgtgggtcttgctcacagaaaattaaaaggttttgtctcttacgctgaaa

SEQ ID NO: 8 - protein, 405 aa, Sus scrofa
MAANVQASKSTPNHVIFKKTSRDKSVTIYLGKRDYIDHVDQVEPVDGVVLVDPELVKGKRVYVSLTCAFRYGQEDIDVIGLSFRRDLYFSQVQVFPPVGAASQTKLQESLIKKLGGHTYPFLLTFPDYLPCSVMLQPAPQDVGKCCGVDFEVKAFATDSADTGEDKIPKKSSVRLLIRKVQHAPLEMGPQPHAEAAWQFFMSDKPLHLTVSLSKELYYHGEPIPVTVTVTNNTEKTVKKIKALVEQVANVVLYSSDYYIKPVATEETQQKVPPNSTLTTTLTLVPLLANNRERRGIADGKIKHEDTNLASSTIIKEGMDKTIMGILVSYQIKVQLTVSGLLGELTSSEVATEVPFRLMHPQPEDPEAMDSFQDENFVFEEFARQNLKDAGELKEEKTDQEAAAHE